(19) United States
(12) Patent Application Publication
(10) Pub. No.: US 2007/0099876 A1
(43) Pub. Date: May 3, 2007

(54) Target directed chemotherapy of tumours of the sexual organs

(76) Inventor: Heinz Forster, Kadenbach (DE)

Correspondence Address: J C Patents, 4 Venture, Suite 250, Irvine, CA, 92618, US

(21) Appl. No.: 10/558,973

(22) PCT Filed: May 18, 2004
(86) PCT No.: PCT/EP04/05333

§ 371 Date: Oct. 31, 2006

(30) Foreign Application Priority Data
May 30, 2003 (DE) 103 24 496.4

(51) Int. Cl. A61K 31/655; A61K 31/381; C07J 1/00

(52) U.S. Cl. 514/150; 514/443; 534/551

(57) ABSTRACT

The invention relates to dialkyltriazene-bearing estrogens and anti-estrogens that are suited for use as chemotherapeutic drugs for treating carcinomas of the sexual organs of humans and animals.

### BACKGROUND OF THE INVENTION

1. Field of the Invention

The present invention relates to novel compounds based on estrogens and anti-estrogens which are suitable as chemotherapeutics against tumours, methods for their preparation and their use for the treatment of diseases, especially of cancer.

2. Description of the Related Art

Healthy cells, cancer cells and cells of the metastases of sexual organs contain estrogen receptors (=cytoplasmic proteins), see “The nuclear receptor ligand-binding domain: structures and function” in Curr. Opin. Cell Biol. 10, 384-391 (1998). The OH groups of the sex hormones possess the ability to bind to the estrogen receptors, therefore natural and synthetic female sexual hormones (estrogens) and their antagonists (anti-estrogens) possess an affinity for the tissues of the sexual organs (mamma, uterus, ovaries, prostate).

Estrogen receptors accumulate especially intensely in the cancer cells of tumours of the sexual organs, e.g. in mammary tumours and their metastases, (E. v. Angerer, The estrogen receptor as a target for rational drug design, pages 5, 49 and 137, Springer-Verlag, Heidelberg 1995). Attempts have previously been made to use estrogen receptors as targets for active agents by coupling, for example, the natural female sex hormone estradiol or the synthetic hormone diethylstilbestrol with an active group, e.g. with a nitrogen lost functionality, in the hope that the estrogen based molecule would carry the active group into the tumour and which then could destroy the tumour (G. Leclercq, Breast Cancer—Experimental and Clinical Aspects, 287-293, Pergamon, Oxford 1980; H. Hamacher, Potentielle Antineoplastika III, Arch. Pharm. 311, 184-195, Verlag Chemie, Weinheim 1978). However, all such attempts failed (E. v. Angerer, loc. cit., 155).

For the development of active agents against tumours of the human sexual organs, an animal model closely similar to human tumours is needed, so that truly significant test results can be accomplished. The known “Huggins tumours” (C. Huggins et al., Rapid induction of mammary carcinoma in the rat and the influence of hormones, J. Exper. Med. 109, 25 (1959)) can be generated and combated easily, e.g. by endocrine manipulations and the usual chemotherapy (e.g. with endoxan®=cyclophosphamide monohydrate). In contrast, the tumours generated by benzidine in female Wistar rats require more time for induction and are (like human mammary tumour) only up to about 50% hormone dependent; they (like human mammary tumour) cannot be influenced effectively by chemotherapeutics and other methods of treatment in the art. Therefore, we have found the desired suitable animal model in Wistar rats with mammary tumours induced by benzidine.

It was now found surprisingly, that estrogens and anti-estrogens bearing dialkyltriazenyl groups cause their degeneration after application to rats with mammary tumours. Therapy directed selectively to mammary tumour tissue does not result in the side effects known from the art, such as damaging of the bone marrow and the intestine epithelium. Dialkylphenyltriazenes are known as general non-selective cytostatics; see Proc. Soc. Exper. Biol. Med. 90, 484 (1955); but similar to alkylating agents (endoxan) they intervene indiscriminately in the proliferation of all body cells.

Moreover 4-(3,3-dimethyl-1-triazenyl)-3-methoxy-estra-1,3,5(110)-trien-17-one is known as an intermediate for the preparation of a fluorine compound (exchange of triazenyl with fluorine); see J. Org. Chem. 46 (12), 2520-2528 (1981).

### SUMMARY OF THE INVENTION

Thus, an object of the invention is estrogens and anti-estrogens, in which each molecule is core-substituted with at least one dialkyltriazenyl group, with the exception of 4-(3,3-dimethyl-1-triazenyl)-3-methoxy-estra-1,3,5(10)-trien-17-one.

The terms “estrogens” and “anti-estrogens” within the spirit of the invention comprise both natural and synthetic estrogen and anti-estrogen active compounds. As estrogen and anti-estrogen active compounds, respectively, the substitution of which with dialkyltriazenyl groups results in the compounds according to invention, basically all compounds are suitable in which the “receptor occupancy ratio”, which is obtained with 10 mg/l testing substance in the “evidence of competitive inhibition against 6,7-ditritium-estradiol on the estrogen receptor” described below, is at maximum 0.9, preferably at maximum 0.7, especially preferably at maximum 0.3.

The term ““core-substituted” within the spirit of the invention relates to one or more aromatic rings of the estrogen and anti-estrogen basic compounds.

Without being bound to a particular theory, the success according to the invention may be explained in the current terms as follows: the triazenyl groups can obviously bind to estrogen receptors like OH groups. For bringing the effective groups to the target, i.e. the tumour tissue, the estrogen and anti-estrogen molecule moiety is used as a carrier, which imparts a hormone like specificity to the compounds according to the invention. Within the cancer cells the effective groups cause the oncolysis of the cancer cells.

The compounds according to the invention can be imparted with some desired characteristics by the introduction of certain groups; for example, the degree of water solubility of the compounds can be controlled as desired within a broad range by introducing hydrophilic groups. Alkaline or ammonium salts of compounds according to invention with hydrophilic groups are well soluble in water.

One advantage of the compounds with hydrophilic groups according to the invention is that in comparison to the estrogen receptors, they can be accumulated in an excess within the cancer cells in amounts required for therapy and that the excess can be eliminated rapidly from the body. Thereby, toxic side effects are minimized.

Due to the hormone like specificity of the compounds according to the invention relatively small amounts are needed.

As carriers according to the invention estrogen and anti-estrogen derivatives, such as for example, from the groups steroids, stilbenes, hexestroles, phenyl-1,2-bis(2,6-dichloro-phenyl)-1,2-bis(ethylenaminoethanes), triphenylethylenes, phenylbenzofuranes, phenylbenzothiophenes, which are especially substituted in position 3 by a benzoyl group, 4,5-bis-phenyl-imidazoles, 2,3-diarylpiperazines and 4,5-phenyl-2-imidazolines can be used. Some of the exemplary carrier types will be discussed in the following.

Compounds according to invention comprise for example steroidtriazenes of the formula 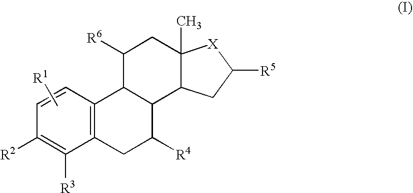
wherein
R¹ is hydrogen, N═N—NR^{7A}R^{7B}, O(CR⁸R⁹)ₙCO₂H, CO₂H or SO₃H,
R² is OH, OCH₃, N═N—NR^{7A}R^{7B} or O(CR⁸R⁹)ₙCO₂H,
R³ is hydrogen, N═N—NR^{7A}R^{7B}, O(CR⁸R⁹)ₙCO₂H, CO₂H or SO₃H,
R⁴ and R⁶ are independently from each other hydrogen, O(CR⁸R⁹)ₙCO₂H, (CR⁸R⁹)ₙCO₂H or C₆H₄OCH₂CO₂H and
R⁴ is moreover (CH₂)₁₀CON(C₁-C₄-alkyl)₂,
R⁵ is hydrogen or OH,
R^{7A} and R^{7B} are independently from each other alkyl,
R⁸ and R⁹ are independently from each other hydrogen, methyl or ethyl,
X is CO, CHOH or C(OH)—C≡CH and
n is an integer from 1 to 10
with the condition, that only one of the residues R¹ to R³ represents N═N—NR^{7A}R^{7B}, and their salts, solvates and solvates of these salts.

The steroidtriazenes I can be obtained for example by diazotization of amino derivatives of the formula 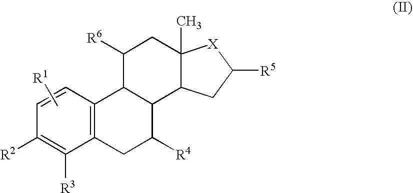
wherein one of the residues R¹ to R³ is NH₂ and the remaining symbols R¹ to R⁶ and X have the meanings given in the legend for formula I, or their salts and by reaction of the resulting diazonium salts of the formula
[R—N₂]⁺Y⁻ (III)
wherein
R is the formula II, in which one of the residues R¹ to R³ is (N₂)⁺, and the other variables have the meanings given in the legend for formula II, and Y⁻ is an acid anion, with dialkylamines and if necessary by releasing the acids from the obtained salts.

By using for example 2-amino-3-carboxymethoxy-estradiol as starting material the reaction can be illustrated by the following formula scheme 1:

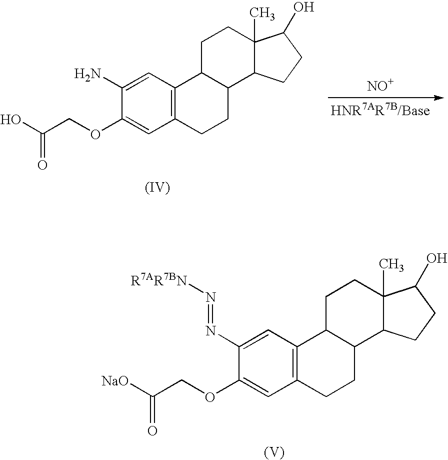

The aminosteroids used as starting materials are either known or can be prepared analogously to known preparation methods.

Examples for aminosteroids II comprise for example:

1-amino-3-oxyacetic-estradiol, 2-amino-3-oxyacetic-estradiol, 4-amino-3-oxyacetic-estradiol, 1-amino-3-oxyacetic-estrone, 2-amino-3-oxyacetic-estrone, 4-amino-3-oxyacetic-estrone, 1-amino-3-methoxy-estradiol, 2-amino-3-methoxy-estradiol, 4-amino-3-methoxy-estradiol, 1-amino-3-methoxy-estrone, 2-amino-3-methoxy-estrone, 4-amino-3-methoxy-estrone, 1-amino-3-oxyacetic-estriol, 2-amino-3-oxyacetic-estriol, 4-amino-3-oxyacetic-estriol, 1-amino-3-oxyacetic-ethinyl-estradiol, 2-amino-3-oxyacetic-ethinyl-estradiol, 4-amino-3-oxyacetic-ethinyl-estradiol, 2-amino-4-sulfonic-estradiol, 4-amino-2-sulfonic-estradiol, 2-amino-4-sulfonic-estrone and 4-amino-2-sulfonic-estrone.

By replacement of the OH group in estrone with a triazenyl group (see example 11) a potent cancer chemotherapeutic is obtained, which shows a very good effect at low dosage against mammary carcinoma of the rat. It can be concluded therefore, that the triazenyl group can surprisingly replace the essential OH group as the adhesion group at the estrogen receptor. If the maximum bond characteristics of the OH group (or of its ethers) are to be used at the estrogen receptor for anti-tumour effect, the triazenyl group is preferred introduced according to the invention in position 2 or 4.

As already mentioned above it can be desirable to impart the compounds according to the invention with hydrophilic groups for purposes of water solubility. Sulfonate and carboxylate groups as well as C₁-C₆ residues bearing suchlike groups are preferred as hydrophilic groups. Even if several positions come into question for an additional substitution, substitutions can be carried out especially easily at the aromatic ring—by for example core-sulfonation or etherification of phenolic hydroxyl groups.

Where the position of substitution is concerned the knowledge, that estrone, estradiol or ethinylestradiol have a strong 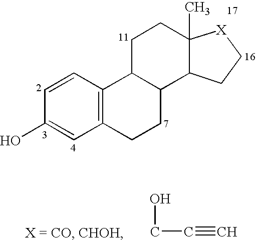
affinity to the estrogen receptor even when large substituents are present in the positions 2, 4, 7 and 11 (P. W. Jungblut et al., Hormon-Rezeptoren, Kolloquium der Gesellschaft für physiologische Chemie vom 05.-08.04.1967 in Mosbach/Baden; M. Görlich, Arch. Geschwulstforschung 37/2, 161-170 (1971)) can be a guide line. Hence, these positions are also preferred for substitutions.

For example, in the position 3 a carboxyalkoxy group, e.g. a carboxymethoxy group can be located as solubilizing group [(prodrug) as salt]. If in one of the positions 2 or 4 the triazenyl group is located, then the second position (4 or 2) can bear a solubilizing group, e.g. the salt of a carboxyalkoxy or of a sulfonic acid group. The positions 7 and 11 can be used for further substituents and can bear for example additional solubilizing groups (e.g. carboxyalkoxy groups); thus, the possibility exists to achieve desired selectivities by introducing substituents if necessary.

Compounds, in which the phenolic OH group is etherified act as prodrug, i.e. it can be expected, that the adhesion at the estrogen receptor is maintained by a carboxymethoxy group in position 3; see e.g. mestranol, see E. Mutschler, Arzneimittelwirkungen, Lehrbuch der Pharmakologie und Toxikologie, page 368, Wissenschaftliche Verlagsgesellschaft, Stuttgart 1997. As in the preparation of chemotherapeutics, in the present case it is only important that the active agent binds to the estrogen receptor, it is not important if the carrier is an estrogen or an anti-estrogen.

The estrogenic steroids are exemplary for how highly specific and highly effective active agents against mammary carcinomas, which bind optimally to the estrogen receptor, can be synthesized from estrogens by the introduction of a dialkyltriazenyl group. It has to be assumed further, that such active agents are effective against all carcinomas of the sexual organs (uterus, ovaries, prostate) which contain estrogen receptors, due to their bonding at the estrogen receptor.

Stilbenes: Cancer Chemotherapeutics Derived from Stilbenes, Diethylstilbestrol and Hexestrol

Further compounds according to the invention are e.g. cis- and trans-stilbenes and hexestroles of the formula 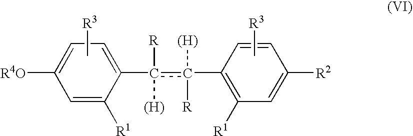
wherein
R is hydrogen, methyl or ethyl,
R¹ is hydrogen, chlorine, methyl, ethyl, CH₂CO₂H, CH(CH₃)CO₂H, OCH₂CO₂H, OCH(CH₃)CO₂H or SO₃H,
R² is OH, OCH₃, OCH₂CO₂H, OCH(CH₃)CO₂H or N═N—NR^{7A}R^{7B},
R³ is hydrogen, chlorine, preferred in position 6, or N═N NR^{7A}R^{7B},
R⁴ is hydrogen, methyl, ethyl, CH₂CO₂H or CH(CH₃)CO₂H and
R^{7A} and R^{7B} are independently from each other alkyl
with the condition, that either R² or R³ represents N═N—NR^{7A}R^{7B}, and the dashed bonds indicate, that the compounds comprise both ethane as well as ethylene derivatives, and their salts, solvates and solvates of these salts.

These compounds VI can be obtained for example by diazotization of amino derivatives of the formula 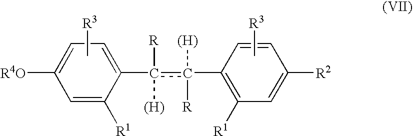
wherein
R³ is hydrogen or NH₂ and
R⁴ is hydrogen, methyl, ethyl, CH₂CO₂H or CH(CH₃)CO₂H
with the condition, that either R² or R³ represents NH₂, and R, R¹, R², R^{7A}, R^{7B} and the dashed bonds have the meanings given in the legend for formula VI, or their salts and by reaction of the resulting diazonium salts of the formula
[R⁵—N₂]⁺Y⁻ (VIII)
wherein
R⁵ is the formula VII, in which one of the both residues R² and R³ is (N₂)⁺, respectively, the other variables have the meanings given in the legend for formula VII, and Y⁻ is an acid anion, with dialkylamines and if necessary by releasing the acids from the obtained salts.

By using for example 3,3′-diamino-diethylstilbestrol (H. Hamacher, Potentielle Antineoplastika III, Arch. Pharm. 311, 184-195, Weinheim 1978) as starting material the reaction can be illustrated by the following formula scheme 2: 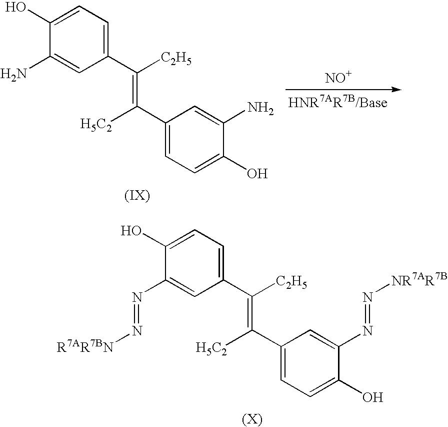
With further reaction of 3,3′-di-(dialkyltriazenyl)-diethylstilbestrol with 1 or 2 equivalents of an alkylating agent of the formula
R⁶—Y (XI)
wherein
R⁶ is C₁-C₃-alkyl, CH₂CO₂CH₃ or CH(CH₃)CO₂CH₃ and
Y is a cleavage group for alkylating agents
the corresponding alkylation products can be obtained; see the following formula scheme 3: 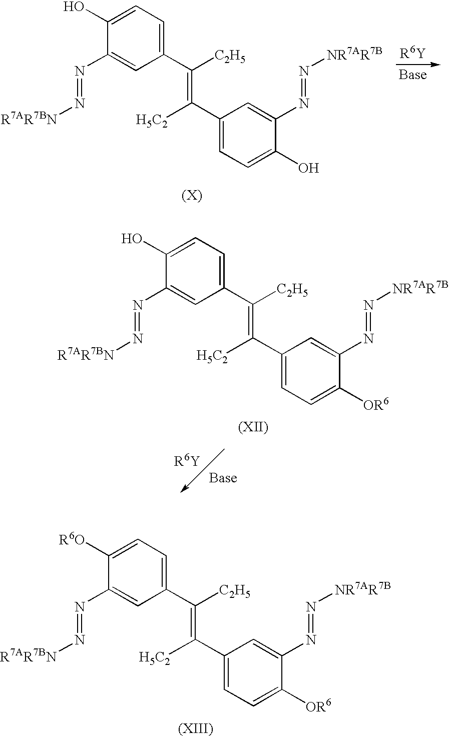
If R⁶ in the above formula scheme 3 is for example CH₂CO₂CH₃, then the disodium salt can be obtained from the bis-ester by basic hydrolysis, for example with NaOH (formula scheme 4):

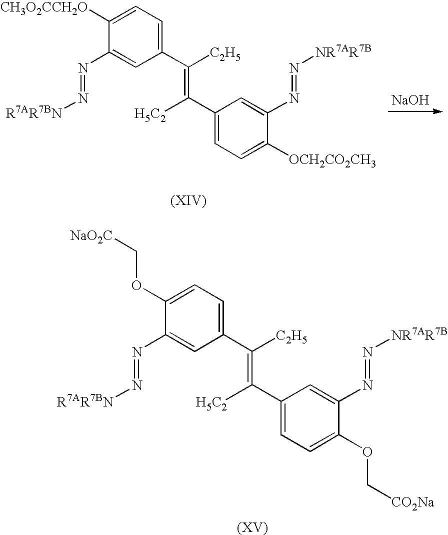

Other compounds according to the invention are for example triphenylethylene derivatives of the formula 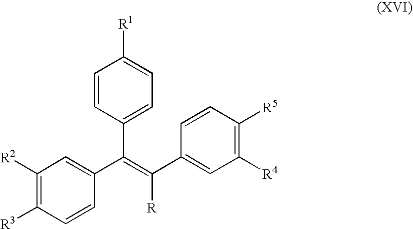
wherein
R is hydrogen, chlorine, chlormethyl or ethyl,
R¹ is OCH₂CO₂H or OCH(CH₃)CO₂H,
R² and R⁴ are independently from each other hydrogen, SO₃H or N═N—NR^{7A}R^{7B},
R³ and R⁵ are independently from each other hydrogen, OH, OCH₃, OCH₂CO₂H, OCH(CH₃)CO₂H or N═N—NR^{7A}R^{7B} and
R^{7A} and R^{7B} are independently from each other alkyl
with the condition, that only one of the residues R² to R⁵ represents N═N—NR^{7A}R^{7B}, and their salts, solvates and solvates of these salts.

The compounds XVI according to the invention can be obtained for example by diazotization of amino derivatives of the formula 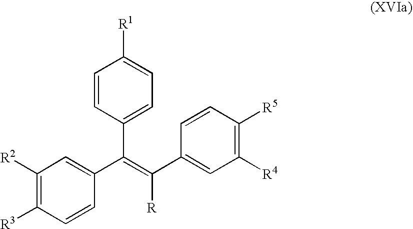
wherein
R² and R⁴ are independently from each other hydrogen, NH₂ or SO₃H,
R³ and R⁵ are independently from each other hydrogen, NH₂, OH, OCH₃, OCH₂CO₂H or OCH(CH₃)CO₂H and
R and R¹ have the meanings given in the legend for formula XVI, with the condition, that only one of the residues R² to R⁵ represents NH₂,
or of their salts and by reaction of the resulting diazonium salts of the formula
[R⁶—N₂]⁺Y⁻ (XVII)
wherein
R⁶ represents the formula XVIa, in which one of the residues R² to R⁵ is (N₂)⁺ and the other variables have the meanings given in the legend for formula XVIa, and Y⁻ is an acid anion, with dialkylamines and if necessary by releasing the acids from the obtained salts.

By using for example the amino compound XVIII as starting material the reaction can be illustrated by the following formula scheme 5:

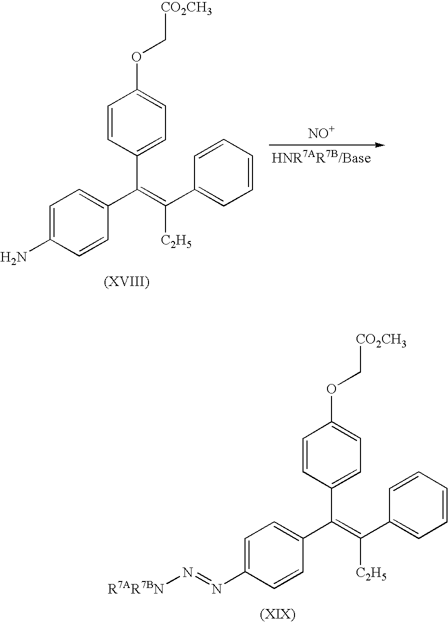

By basic hydrolysis of the methyl ester XIX the sodium salt XX can be obtained according to reaction scheme 6:

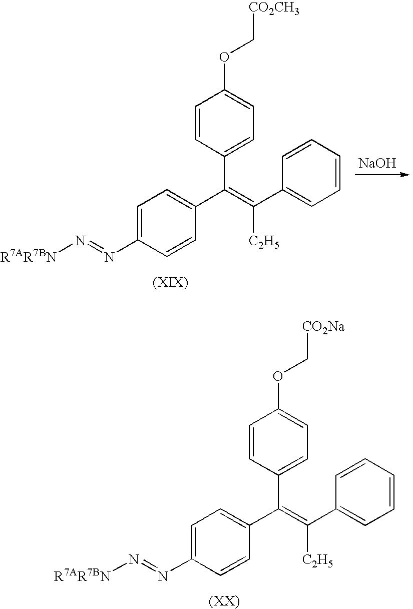

In the exemplary way shown above cancer chemotherapeutics can be prepared from any estrophilic compounds by introducing the triazenyl group, e.g.:

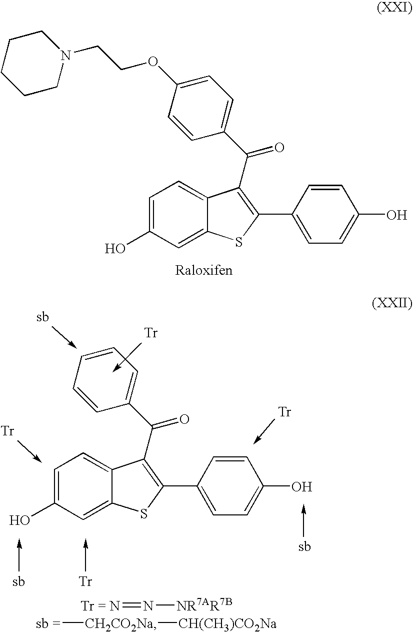

The arrows indicate preferred positions for triazenyl—(Tr) and solubilizing groups (sb).

Hence, a further object of the invention is a method of preparing estrogens and anti-estrogens, in which each molecule is core-substituted with at least one dialkyltriazenyl group, whereupon at least one dialkyltriazenyl substituent is introduced in one or more aromatic rings of an estrogen or anti-estrogen active compound, with the exception of preparing 4-(3,3-dimethyl-1-triazenyl)-3-methoxy-estra-1,3,5(10)-trien-17-one.

Within the scope of the invention physiologically acceptable salts are preferred as salts.

Physiologically acceptable salts, preferred physiologically acceptable salts of the compounds I, II, VI, VII, XVI and XVIa, comprise salts of usual bases, such as for example alkaline metal salts (e.g. sodium and potassium salts), earth alkaline salts (e.g. calcium and magnesium salts) and ammonium salts, which are derived from ammonia or organic amines with 1 to 16 C atoms, such as for example ethylamine, diethylamine, triethylamine, ethyldiisopropylamine, monoethanolamine, diethanolamine, tris-hydroxyethylamine, dicyclohexylamine, dimethylaminoethanol, procaine, dibenzylamine, N-methylmorpholine, dihydroabietylamine, arginine, lysine, ethylendiamine and methylpiperidine.

Within the scope of the invention such forms of the compounds are referred to as solvates, which form a complex with solvent molecules by coordination in solid or liquid phase. Hydrates are a special form of the solvates, in which the coordination takes places with water. Alkyl per se and “alkyl” and “alk” in dialkylamine and carboxyalkoxy represent a linear or branched alkyl residue with generally 1 to 6, 1 to 4 or 1 to 3 C atoms, for example for methyl, ethyl, n-propyl, isopropyl, tert.-butyl, n-pentyl and n-hexyl.

Diazotizations are known; see e.g. Organikum, 10. Ed., VEB Deutscher Verlag der Wissenschaften, Berlin 1971, 580-600.

Acid anions within the scope of the invention are especially the anions of mineral acids, carboxylic acids and sulfonic acids, e.g. salts of the hydrochloric acid, hydrobromic acid, sulfuric acid, phosphoric acid, methanesulfonic acid, ethanesulfonic acid, toluolsulfonic acid, benzosulfonic acid, naphthalindisulfonic acid, acetic acid, propionic acid, lactic acid, tartaric acid, malic acid, citric acid, fumaric acid, maleic acid and benzoic acid.

Cleavage groups for alkylating agents within the scope of the invention comprise for example chloride, bromide and sulfate.

A further object of the invention are estrogens and anti-estrogens, in which each molecule is core-substituted with at least one dialkyltriazenyl group, for the treatment of diseases.

A further object of the invention is the use of estrogens and anti-estrogens, in which each molecule is core-substituted with at least one dialkyltriazenyl group, against tumours of the sexual organs of humans and animals.

A further object of the invention is a method of combating tumours of the sexual organs of humans and animals by application of a sufficient amount of at least one compound from the group of estrogens and anti-estrogens, in which each molecule is core-substituted with at least one dialkyltriazenyl group.

A further object of the invention is the use of estrogens and antiestrogens, in which each molecule is core-substituted with at least one dialkyltriazenyl group, for the preparation of pharmaceuticals against tumours of the sexual organs of humans and animals.

A further object of the invention are pharmaceuticals containing at least one compound of the group of estrogens and antiestrogens, in which each molecule is core-substituted with at least one dialkyltriazenyl group, if necessary together with one or more pharmacologically acceptable adjuvants or substrates, as well as their use for the above mentioned purposes.

The compounds according to the invention can be effective systemically and/or locally. For this purpose they can be applied by suitable route, e.g. by a oral, parenteral, pulmonal, nasal, sublingual, lingual, buccal, rectal, transdermal, conjunctival, otic route or as an implant.

For these routes of application the active agent can be administered in suitable forms.

Suitable for oral application are known forms of application, which deliver the active agent rapidly and/or in a modified form, such as for example pills (non coated as well as coated pills), e.g. pills or coated pills with coatings resistant to gastric juice), capsules, sugar-coated pills, granulates, pellets, powders, emulsions, suspensions, solutions and aerosols.

Parenteral application can be carried out by avoiding a resorption step (by intravenous, intraarterial, intracardial, intraspinal or intralumbal route) or by involving a resorption step (by intramuscular, subcutaneous, intracutaneous, percutaneous, or intraperitoneal route). Suitable for parenteral application forms of application are, for example, injection and infusion formulations in form of solutions, suspensions, emulsions, lyophilisates and sterile powders.

Suitable for other routes of application are, for example, inhalation medicament formulations (for example, powder inhalators, nebulizers), nasal drops/solutions, sprays; tablets or capsules to be applied by a lingual, sublingual or buccal route, suppositories, ear and eye preparations, vaginal capsules, aqueous suspensions (lotions, agitation mixtures), lipophilic suspensions, ointments, creams, milk, pastes, dispersing powders or implants.

The active agents can be transformed into the mentioned forms of application in a known manner. This can be effected by using inert, non toxic, pharmaceutically suitable adjuvants. Included are, for example, substrates (e.g. microcrystalline cellulose), solvents (e.g. liquid polyethylene glycols), emulsifiers (e.g. sodium dodecylsulfate), dispersing agents (e.g. polyvinylpyrrolidone), synthetic and natural biopolymers (e.g. albumin), stabilizers (e.g. antioxidants such as ascorbic acid), colorants (e.g. inorganic pigments such as iron oxides) or flavoring and/or odorant agents.

Generally it is to be recommended to administer in the case of parenteral application amounts of about 1 to 20 mg/kg, preferred about 2.5 to 10 mg/kg body weight, to achieve effective results. In the case of oral application the amount is about 1 to 70 mg/kg, preferred about 1 to 30 mg/kg body weight.

Nevertheless it can be required, if necessary, to deviate from the mentioned amounts depending from body weight, route of application, individual reaction to the active agent, type of formulation and point of time or time period, in which the application takes place. Thus, it may be sufficient in some cases, to apply less than the above mentioned minimum amount, while in other cases the mentioned maximum amount has to be exceeded. In the case of the application of greater amounts it can be advisable to portion them in several individual doses over the day.

### DESCRIPTION PREFERRED EMBODIMENTS

Generation of Test Tumors

20 female Wistar rats are treated with benzidine. The first benzidine dose is 150 mg/kg; then the doses are reduced to weekly 100 mg/kg. Then doses per 75 mg/kg follow within a gap of 14 days. There from a total dose of 1.225 g/kg results.

In total 29 mammary carcinomas are generated. The average life expectancy of the benzidin treated Wistar rats is 325 days.

After the first tumour is generated (about 0.5 g) the benzidine application is stopped. Then the mammary carcinomas are growing relatively fast. The tumour size is determined tactually; experienced experimenters can determine it precisely to 0.1 g. Tumours of more than 0.5 g are used for the tests.

Evidence of Competitive Inhibition Against 6,7-Ditritium-Estradiol on the Estrogen Receptor by Compounds According to Invention

In the tests 6,7-ditritium-estradiol is used with a specific activity of 0.5 Ci/mmol.

Tissue samples of 0.7 mm thickness and 0.5 cm² area from uterus and mammary carcinoma tissue of the rat are prepared with a tissue cutter. The carcinoma should have a minimum weight of 1.5 g for tissue samples.

Treated Tissue Samples

Tissue samples from uterus and mammary carcinoma are treated in physiological common salt solution with concentrations of 5, 10, 15, 20, 35 and 50 mg/l of compound according to invention. Then all of the tissue samples are washed and incubated again for 1 hour in physiological common salt solution, which contains 10⁻⁹ mol/l 6,7-ditritium-estradiol=physiological concentration.

Control

Tissue samples as above, but not treated, are incubated for 1 hour with physiological common salt solution.

After the incubation all tissue samples are washed and placed into 4% formol. Then they are dried, balanced and combusted. The ash is dissolved in scintillation fluid and the activity is determined in a fluid scintillation counter. The number of the light flashes per mg of ash dry matter in a defined period of time is a measure of the incorporated amount of 6,7-ditritium-estradiol and hence an indirect measure of the number of the estrogen receptors, which have been replaced with the compound according to invention. The ratio between the number of light flashes obtained from rats treated according to invention and the number of light flashes obtained from untreated rats is referred to for purposes of this invention as “receptor occupancy ratio” (means: occupied by estradiol). Hence, it applies, that the smaller the ratio, the stronger the compound according to invention has occupied the estrogen receptors; the estradiol used in the test can only occupy the receptors, which are not occupied (by a compound according to invention).

It is in the nature of the examined tissues, that the number of the estrogen receptors per tissue sample varies within a certain range. Thus, for example the number of the estrogen receptors of adjacent tissue samples of a mammary tumour (due to different density of the tumour tissue) can be different. Therefore it is recommended to examine 3 samples at a time and to calculate an average value.

If the amount of compound according to the invention chosen for the test is relatively high, the receptors, which can be occupied by the compounds according to invention, are already occupied by the first amount and an increase does not show a significant effect.

Qualitative Evidence of the Cytotoxic Effect of Compounds According to Invention Against Mammary Tumour Cells

With this thymidine test the tumour growth can be easily checked: if no further thymidine is incorporated into the cell, the tumour growth is stopped.

Untreated rats and rats treated with compounds according to the invention are fed with tritium marked thymidine.

The autoradiogramm shows in the tumour tissue of the untreated control group many marked cells. The cells marked with tritium are identifiable by many black dots in the autoradiogramm (strong silver precipitation on the photo plate). These dots are a sign of the strong growth of the tumour cells and the incorporation of the nucleoside thymidine connected there with.

In the case of the rats treated with compounds according to the invention small tumours disappear slowly, while in the case of large tumours necrosis takes place.

Mammary carcinomas of rats, that are treated for 20 days with 20 mg/kg per day of a compound according to the invention, show after feeding with tritium marked thymidine in the autoradiogramm no further thymidine incorporation, i.e. the growth of the tumour cells is at least stopped.

The given percentages in the following examples are, unless indicated otherwise, weight percentages; parts are weight parts. Solvent ratios, dilution ratios and given concentrations of liquid/liquid solutions refer to the volume in each case.

### EXAMPLES

### Examples for the Preparation of the Starting Materials (Labelled with “A”) and Examples of Preparation

All preparations were examined for purity by thin layer chromatography (silica 60 F 254, Merck, Darmstadt). NMR-spectra were recorded of all starting materials prepared by ourselves and of all compounds according to the invention; they match with the postulated structures.
I. Steroids: Reaction Scheme of the Reactions Carried Out:

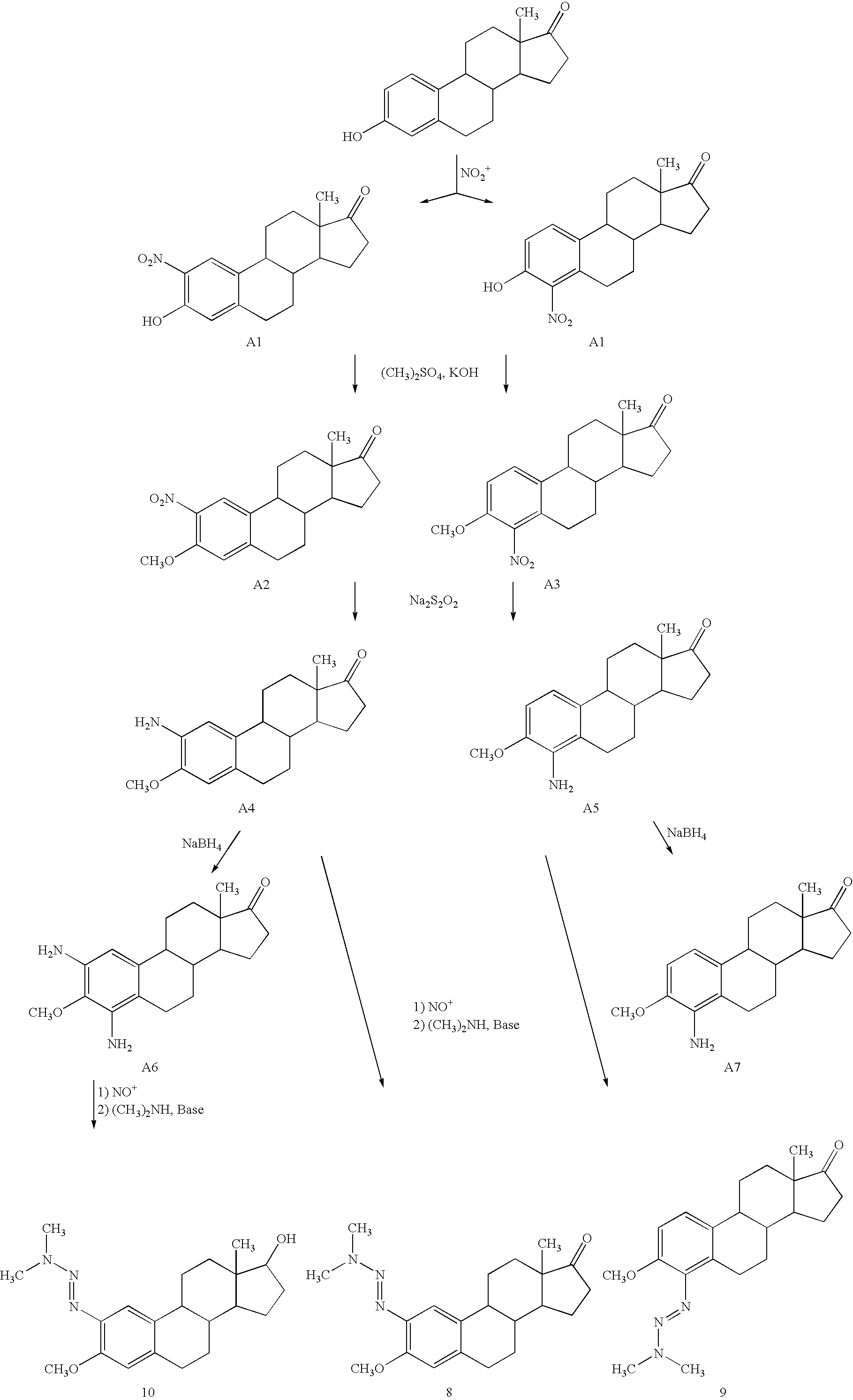

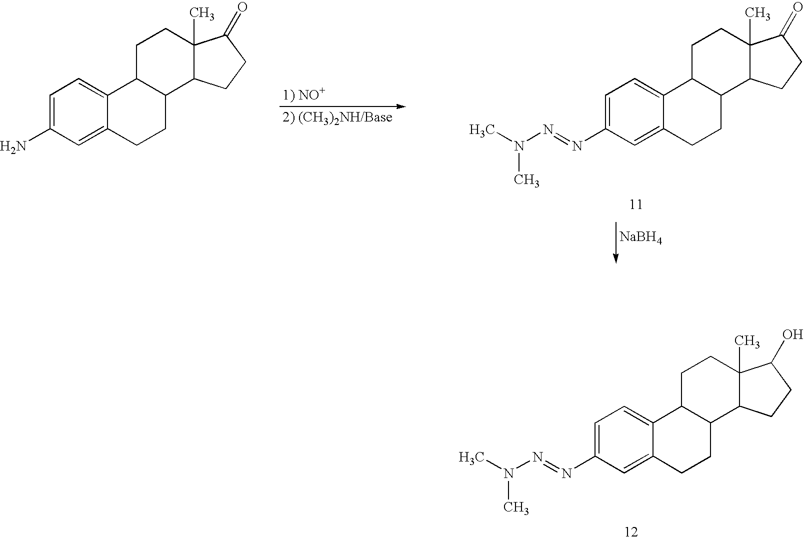

The nitrated estrones, estradiols used as starting materials and the amino compounds prepared there from are either known or can be prepared analogously to known methods (see St. Kraychy, Am. Soc. 81, 1702 (1959)).

### Example A1

### Preparation of 2- and 4-nitroestrone

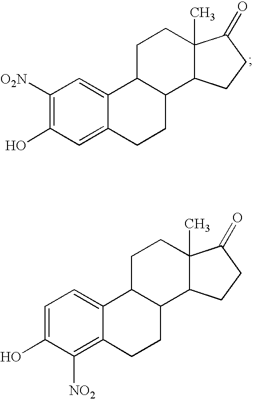

To a solution of 40 g of estrone in 1000 ml of pure acetic acid at 35 to 40° C. under stirring slowly 16.48 ml of conc. nitric acid are added dropwise. It is stirred for 24 hours. 4-Nitroestrone precipitates as light yellow crystals, is extracted by suction and recrystallized from ethanol.

Yield: 9 g of 4-nitroestrone; mp. 270° C.

The filtrate is mixed with 4000 ml of water, the precipitated crude product is extracted by suction and dried (yield: 45 g). The purification is carried out by column chromatography over aluminumoxide (Fa. Woelm), AKT. St. I acidic. The crude product is dissolved in 300 ml of benzene in the heat (max. 15 g) and given with a pipette slowly on the prepared column (height 120 cm, diameter 4.5 cm). Then it is eluted with benzene under DC control. The solution is reduced and the remaining 2-nitroestrone is isolated.

Yield (from 3 columns): 25 g of 2-nitroestrone; mp. 180° C.

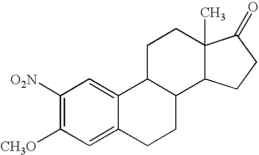

### Example A2

### 2-nitro-3-methoxyestrone

To a solution of 16 g 2-nitroestrone from example A1, 750 ml of ethanol and 750 ml of 10% aqueous potassium hydroxide solution at 35° C. within 6 hours under nitrogen atmosphere 480 ml of dimethylsulfate are added dropwise. It is ensured that the solution remains basic; if necessary 45% aqueous potassium hydroxide solution is added dropwise. It is stirred as long as the solution remains light yellow and basic. Then the solution is cooled to about 5° C., the precipitated product is extracted by suction and washed with diluted aqueous potassium hydroxide solution and water, dried and recrystallized from ethanol/toluene (1:1).

Yield: 15.7 g; mp. 154° C.

### Example A3

### 3-methoxy-4-nitroestrone

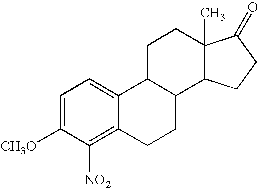

From the compound of example A1 the title compound is prepared according to example A2.

Yield: 2.1 g; mp. 259° C.

### Example A4

### 2-amino-3-methoxyestrone

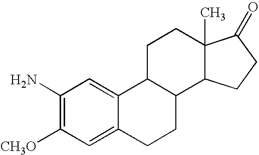

A solution of 5 g of 2-nitro-3-methoxyestrone from example A2, 4 g of sodium thiosulfate, 800 ml of acetone and 160 ml of 0.5 N sodium hydroxide is heated under reflux for 35 minutes, a solution of 3.2 g sodium thiosulfate in 160 ml of 0.5 N sodium hydroxide is added and the resulting solution is heated under reflux for 50 minutes. Then 400 ml of water are added and the acetone is removed under reduced pressure. The obtained suspension is cooled and the crystals are extracted by suction, washed with water, dried and recrystallized from methanol.

Yield: 4.2 g; mp. 155° C.

### Example A5

### 3-methoxy-4-aminoestrone

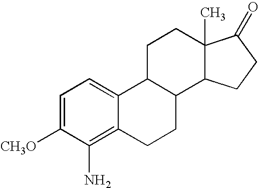

From the compound of example A3 the title compound is prepared according to example A4.

Yield: 0.6 g; mp. 183° C.

### Example A6

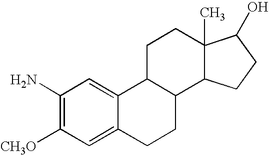

### 2-amino-3-methoxyestrole

A mixture of 1 g of 2-amino-3-methoxyestrone from example A4, 200 ml of methanol and 0.44 g of sodium borohydride is stirred at 40 to 50° C. under DC control until complete reaction (14 hours). Then 4 ml of pure acetic acid are added and the methanol is removed under reduced pressure. The residue is dissolved in diluted hydrochloric acid in the heat, the title compound is precipitated with sodium hydroxide, the suspension is cooled and the precipitated product is extracted by suction and dried.

Yield: 0.6 g; mp. 160° C.

### Example A7

### 3-methoxy-4-aminoestrole

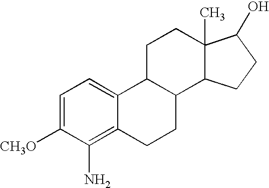

From the compound of example A5 the title compound is prepared according to example A6.

Yield: 0.4 g; mp. 176° C.

### Example 8

### 2-(1,1-dimethyltriazenyl)-3-methoxyestrone

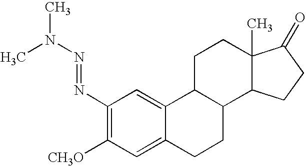

At 0 to 4° C. a solution of 0.55 g sodium nitrite in 3 ml of water is added dropwise to a solution of 2.3 g of 2-emino-3-methoxyestrone from example A4, 160 ml of water and 1.2 ml of conc. (37%) hydrochloric acid. Then the obtained diazonium salt solution is rapidly transferred at 0 to 4° C. into a solution of 0.95 g sodium carbonate, 1 ml of 40% aqueous dimethylamine solution and 40 ml of water. After a stirring period of 1 hour the solid product is extracted by suction, dried and recrystallized from a little toluene.

Yield: 1.5 g; mp. 168° C.

### Example 9

### 3-methoxy-4-(1,1-dimethyltriazenyl)-estrone

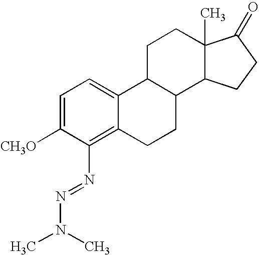

From the compound of example A5 the title compound is prepared according to example 8.

Yield: 1.5 g; mp. 142° C.

### Example 10

### 2-(1,1-dimethyltriazenyl)-3-methoxyestrole

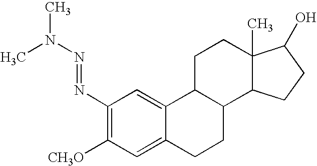

From the compound of example A6 the title compound is prepared according to example 8.

Yield: 3 g; mp. 135° C.

### Example 11

### 3-(1,1-dimethyltriazenyl)-estrone

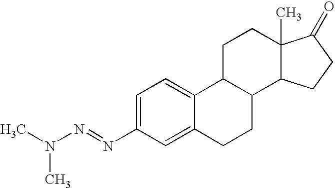

To a solution of 2.7 g of 3-aminoestrone, 40 ml of water and 2 ml of conc. hydrochloric acid (37%) at 0 to 4° C. a solution of 0.7 g of sodium nitrite and 10 ml of water is added dropwise. Then the obtained diazonium salt solution is transferred into a solution of 1.2 g sodium carbonate, 1.2 ml of 40% aqueous dimethylamine solution and 20 ml of water. After a stirring period of 1 hour the title compound is extracted by suction, dried and recrystallized from a little ligroin.

Yield: 2 g; mp. 168° C.

### Example 12

### 3-(1,1-dimethyltriazenyl)-estrole

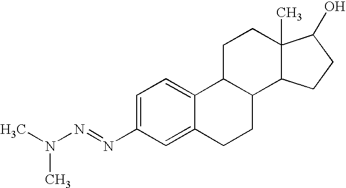

To a solution of 1 g of 3-(1,1-dimethyltriazenyl)-estrone from example 11 in 200 ml of methanol 0.44 g of sodium borohydride are added. The solution is stirred at 40 to 50° C. under DC control until complete reaction (14 hours). Then 4 ml of pure acetic acid are added and the methanol is removed under reduced pressure. The residue is levigated with water, the title compound is extracted by suction and dried.

Yield: 1 g; mp. 138° C.

II. Stilbenes

The stilbenes of the examples were prepared by the Wittig olefin reaction (G. Wittig, Angew. Chem. 68, 505).

In the synthesis of the nitrostilbenes via the Wittig olefin reaction it was found for the first time, that if para-substituted benzaldehydes are exchanged into ortho-substituted ones, the Wittig olefin reaction yields in increasing fractions of cis-stilbene. If the ethylacetate group is located in ortho-position to the aldehyde group, cis-stilbene results in 100%.

The cis-fraction obviously depends from the type and dimension of the substituent at the benzene core of the aldehyde.

The compounds used as starting materials are either known or can be prepared analogously to known methods.

Reaction scheme of the reactions carried out:

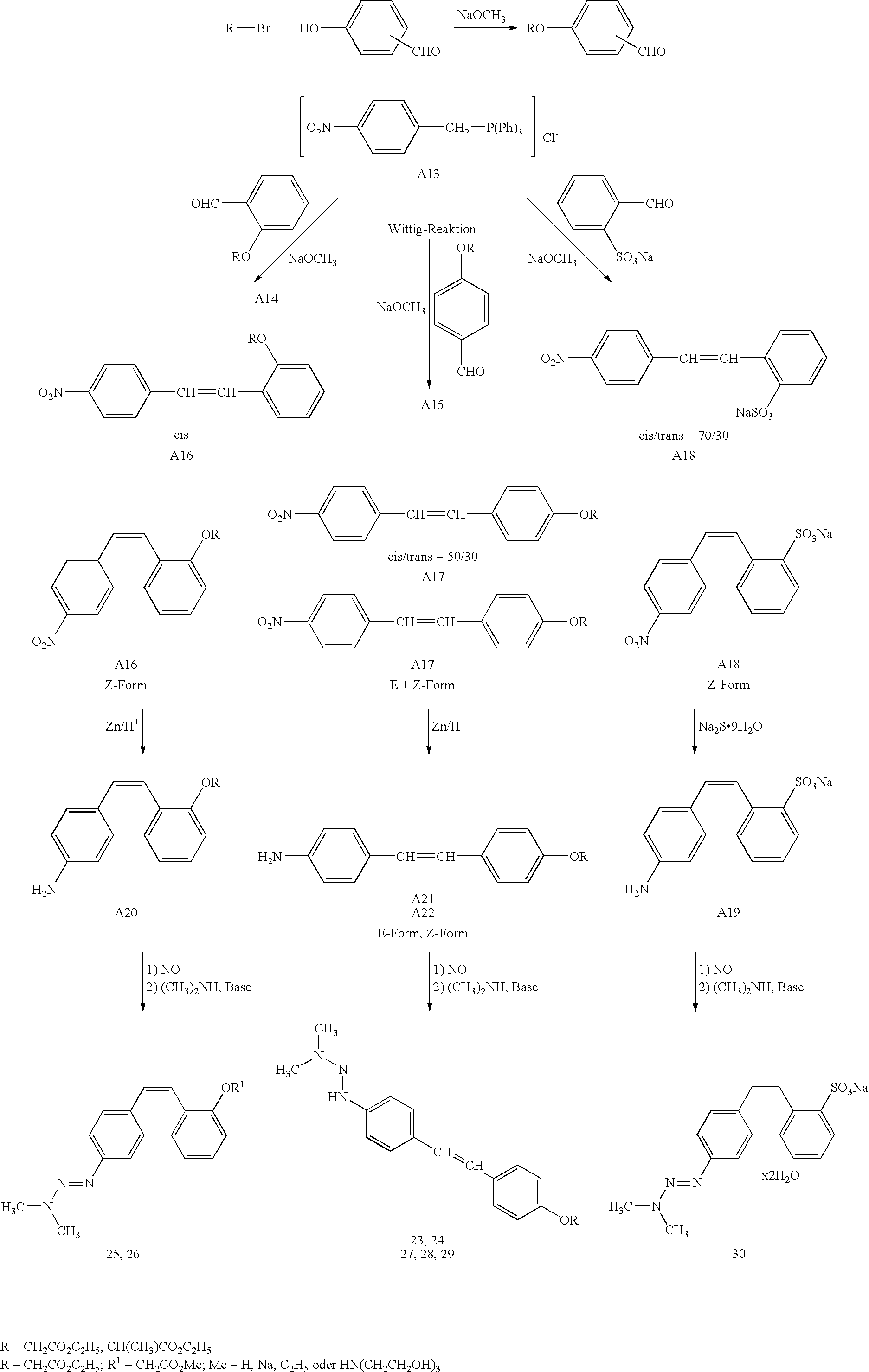

### Example A13

### (4-nitrobenzyl)-triphenylphosphoniumchloride

[O₂N—C₆H₄—CH₂—P⁺Ph₃] Cl⁻

A solution of 263 g (1 mol) of triphenylphosphine, 172 g (1 mol) of 4-nitrobenzylchloride and toluene is stirred for 15 hours at the boiling temperature. The reaction mixture is cooled, the crystals are extracted by suction and washed with toluene.

Yield: 344 g; mp. 280° C.

### Example A14

### (2-formylphenoxy)-ethylacetate

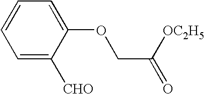

At 50° C. under stirring 167 g (1 mol) of bromoethylacetate are added dropwise to a solution of 122 g (1 mol) of 2-hydroxybenzaldehyde, 1 mol of sodium methylate (in methanolic solution) and 1.5 l of acetonitrile, and the solution is stirred for 7 hours at 70° C. Then the reaction solution is transferred into 2 l of ice water. The separating oil is extracted with 500 ml of dichloromethane, the organic phase is dried over sodium sulfate and the dichloromethane is removed in the vacuum. The remaining oil crystallizes.

Yield: 180 g; mp. 48° C.

### Example A15

### (4-formylphenoxy)-ethylacetate

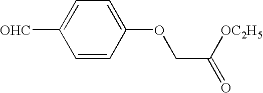

The compound is prepared according to example A14 from 4-hydroxybenzaldehyde.

Yield: 195 g; mp. 43° C.

### Example A16

### {2-[(Z)-2-(4-nitrophenyl)-ethenyl]-phenoxy}-ethylacetate

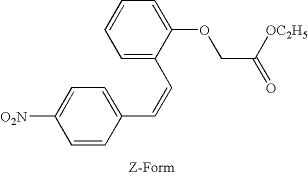

At 0 to 5° C. 218 g of phosphonium salt from example A13 and 0.5 mol of sodium methylate solution are added simultaneously in portions (after respective decolorization) to a solution of 104 g (0.5 mol) of aldehyde from example A14 and 750 ml of ethanol. After decolorization of the reaction solution it is separated from the undissolved. The filtrate is freed from solvent and the residue is levigated with 400 ml of phosphoric-tris-(dimethylamide). At 0° C. triphenylphosphinoxide precipitates from the solution, which is extracted by suction. After addition of 2 l of ice water to the filtrate the precipitating oil is extracted with 3 l of benzene, the solvent is removed, the residue is stirred with 500 ml of isopropanol and after cooling the crystalline product is extracted by suction.

Yield: 105 g of pure cis-compound; mp. 70° C.

### Example A17

### {2-[(E)- and {2-[(Z)-2-(4-nitrophenyl)-ethenyl]-phenoxy}-ethylacetate

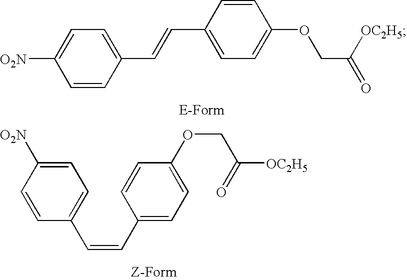

At 0 to 5° C. 218 g of phosphonium salt from example A13 and 0.5 mol of sodium methylate solution are added simultaneously in portions (after respective decolorization) to a solution of 104 g (0.5 mol) of aldehyde from example A15 and 750 ml of ethanol. After decolorization of the reaction solution the precipitated crystals are extracted by suction and recrystallized from ethanol. It is the pure trans-compound.

Yield: 51 g; mp. 118° C.

For isolating the cis-fraction it is proceeded as described in example A16.

Yield: 48 g; mp. 59° C.

### Example A18

### Sodium-2-[(E)- and -2-[(Z)-2-(4-nitrophenyl)-ethenyl]-benzolsulfonate

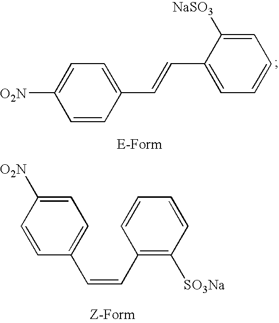

To a solution of 83.2 g (0.4 mol) of sodium-benzaldehyde-2-sulfonate in 500 ml of methanol 0.4 mol of sodium methylate (in methanolic solution) are added. To the resulting solution at 0° C. under stirring a solution of 173 g of phosphonium salt from example A13 and 400 ml of methanol are added dropwise. After decolorization of the reaction solution the solvent is removed under vacuum, the residue is slurred with 300 ml of water and extracted by suction in the cold. The solid product is slurred 1 l of diethylether, extracted by suction, air dried and then boiled with 1.3 l of nitromethane and extracted by suction from the undissolved residue (trans-compound). The cis-compound crystallizes during cooling.

Yield cis-compound: 53 g; mp. 247° C. (from nitromethane).

Yield trans-compound: 20 g; mp. 323° C.

### Example A19

### Sodium-2-[(E)-2-(4-aminophenyl)-ethenyl]-benzolsulfonate

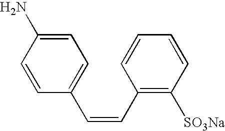

To a solution of 16 g of cis-nitro-compound from example A18 in 60 ml of ethanol at 80° C. a solution of 17.5 g of Na₂Sx9H₂O and 20 ml of water is added dropwise and stirred for 1 hour. Then the solvent is removed in the vacuum and the residue is recrystallized from nitromethane.

Yield: 13 g; mp. >360° C.

### Example A20

### {2-[(Z)-2-(4-aminophenyl)-ethenyl]-phenoxy}-ethylacetate

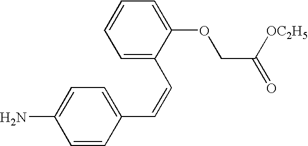

A solution of 16 g of ammonium chloride in 60 ml of water at a maximum of 30° C. under stirring is slowly added dropwise to a mixture of 65.4 g (0.2 mol) of nitro-compound from example A16, 800 ml of acetone and 200 g of zinc dust. Then it is stirred for 20 hours. Then the zinc is extracted by suction and washed with 1 l of acetone in the heat. The acetonic solutions are reduced. The residue is dissolved in 800 ml of water and 25 ml of conc. hydrochloric acid and immediately extracted twice with ethylacetate. Then it is rapidly adjusted with sodium hydroxide to light basic and immediately extracted once again with ethylacetate. The organic phase is dried over sodium sulfate and the solvent is removed under vacuum.

Yield: 55 g, brown oil. The crude product is processed further.

### Example A21

### {2-[(E)-4-(4-aminophenyl)-ethenyl]-phenoxy}-ethylacetate

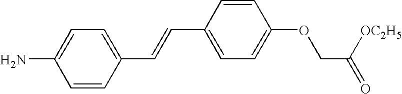

From the trans-nitrostilbene from example A17 the title compound is prepared according to example A20.

Yield: 45 g, yellow crystals; mp. 118° C.

### Example A22

### {2-[(Z)-4-(4-aminophenyl)-ethenyl]-phenoxy}-ethylacetate

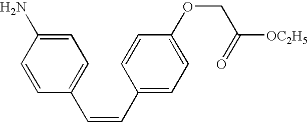

From the cis-nitrostilbene from example A17 the title compound is prepared according to example A20.

Yield: 35 g, yellow oil.

### Example 23

### [4-((Z)-2-{4-[(1E)-3,3-dimethyl-1-triazenyl]-phenyl}-ethenyl)-phenoxy]-ethylacetate

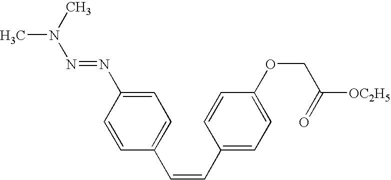

Into a 50° C. hot mixture of 80 ml of water, 600 ml of ethanol and 60 ml of conc. hydrochloric acid are rapidly transferred 71 g of cis-aminostilbene from example A22; the mixture is stirred strongly and cooled rapidly to 0° C. Then immediately a solution of 18 g of sodium nitrite and 70 ml of water are admixed and it is stirred for 1 hour at 0° C. This solution is then rapidly admixed under strong stirring at 0° C. into a mixture of 80 g of 40% aqueous dimethylamine solution, 120 g of sodium carbonate and 1 l of water. It is stirred for 1 hour at room temperature, the reaction product is extracted by suction, dried and further processed as crude product.

Yield: 63 g.

### Example 24

### [4-((E)-2-{4-[(1E)-3,3-dimethyl-1-triazenyl]-phenyl}-ethenyl)-phenoxy]-ethylacetate

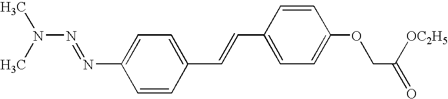

From 0.033 mol of the trans-aminostilbene from example A21 the title compound is prepared according to example 23

Yield: 7 g, mp. 113° C.

### Example 25

### [2-((E)-2-{4-[(1Z)-3,3-dimethyl-1-triazenyl]-phenyl}-ethenyl)-phenoxy]-ethylacetate

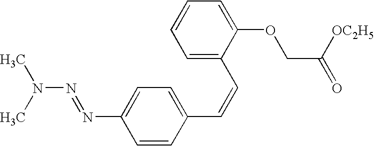

From 0.2 mol of the cis-aminostilbene from example A20 the title compound is prepared according to example 23. The light yellow oil is further processed as crude product.

Yield: 55 g.

### Example 26

### Sodium-[2-((Z)-2-4-[([1E)-3,3-dimethyl-1-triazenyl]-phenyl}-ethenyl)-phenoxy]-acetate

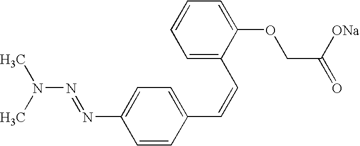

To a boiling solution of 35.3 g of the triazenylstilbenester from example 25 and 850 ml of ethanol 50 g of 10% sodium hydroxide solution is admixed and heated for further 20 minutes under reflux. Then 300 ml of water and 350 ml of a saturated aqueous sodium chloride solution are added. The title compound precipitates, is extracted by suction, dried and recrystallized from acetonitrile.

Yield: 33 g, beige; mp. 72° C.

### Example 27

### Sodium-[4-((Z)-2-{4-[(1Z)-3,3-dimethyl-1-triazenyl]-phenyl}-ethenyl)-phenoxy]-acetate

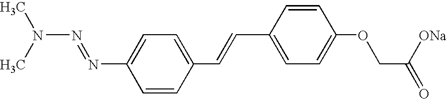

To a boiling solution of 35.3 g of the trans-triazenylstilbenester from example 24 and 850 ml of ethanol 50 g of 10% sodium hydroxide solution is admixed and heated for further 20 minutes under reflux. Then 300 ml of water are added and the precipitated sodium salt is extracted by suction.

Yield: 30 g.

### Example 28

### [4-((Z)-2-{4-[(1Z)-3,3-dimethyl-1-triazenyl]-phenyl}-ethenyl)-phenoxy]-acetic acid

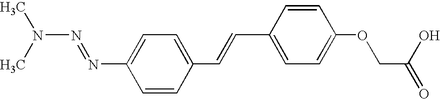

5 g of the compound from example 27 are dissolved in a mixture of 250 ml of phosphoric-tris-(dimethylamide) and 250 ml of water in the heat. It is cooled then to 40° C. and it is extracted by suction. The filtrate is cooled to 30° C. and admixed with 60 ml of pure acetic acid. Under further cooling immediately 50 ml of ice water and 30 ml of pure acetic acid are added. After 10 minutes the title compound is extracted by suction.

Yield: 3 g; mp. 170° C.

### Example 29

### Triethanolammonium-[4-((Z)-2-{4-[(1Z)-3,3-dimethyl-1-triazenyl]-phenyl}-ethenyl)-phenoxy]-acetate

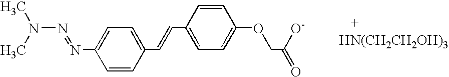

The compound from example 28 is passed to the biological examination as 10% aqueous solution in the form of the triethanolamine salt.

### Example 30

### Sodium-2-((Z)-2-{4-[(1E)-3,3-dimethyl-1-triazenyl]-phenyl}-ethenyl)-benzolsulfonate-dihydrate

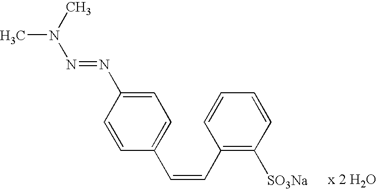

To a solution of 15 g of cis-aminostilbene from example A19, 10 ml of water and 24 ml of conc. hydrochloric acid at 0° C. a solution of 3.5 g of sodium nitrite and 5 ml of water are added dropwise, it is stirred for 10 minutes and the resulting diazonium salt solution is rapidly added dropwise into a solution of 30 g of sodium carbonate, 60 ml of water and 7 g of 40% aqueous dimethylamine solution. It is stirred for 40 minutes, the crystalline reaction product is extracted by suction and recrystallized from acetonitrile.

Yield: 17 g.
III. Triphenylethylene Derivatives

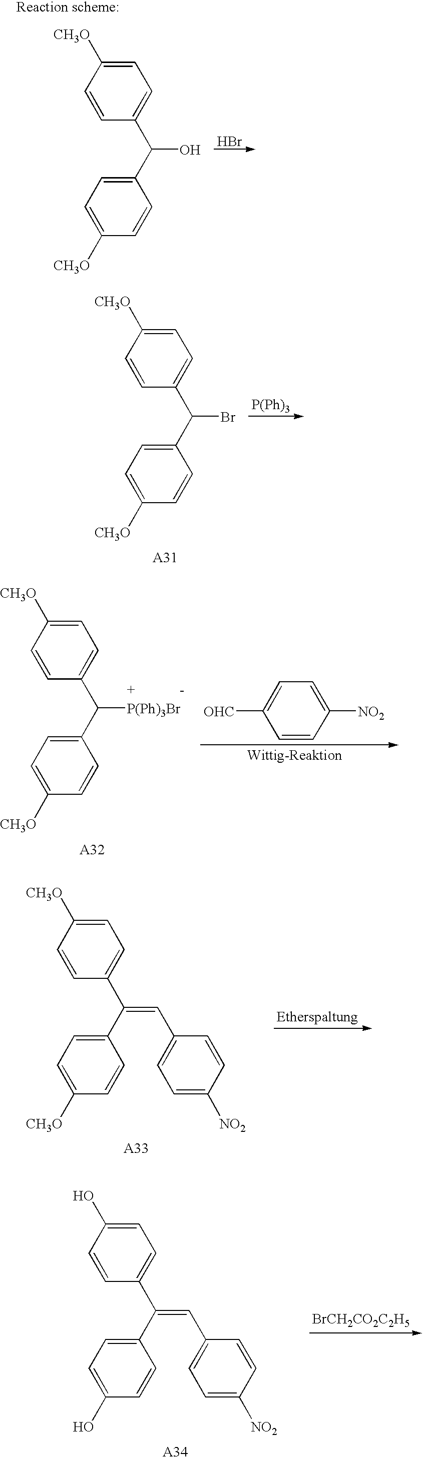

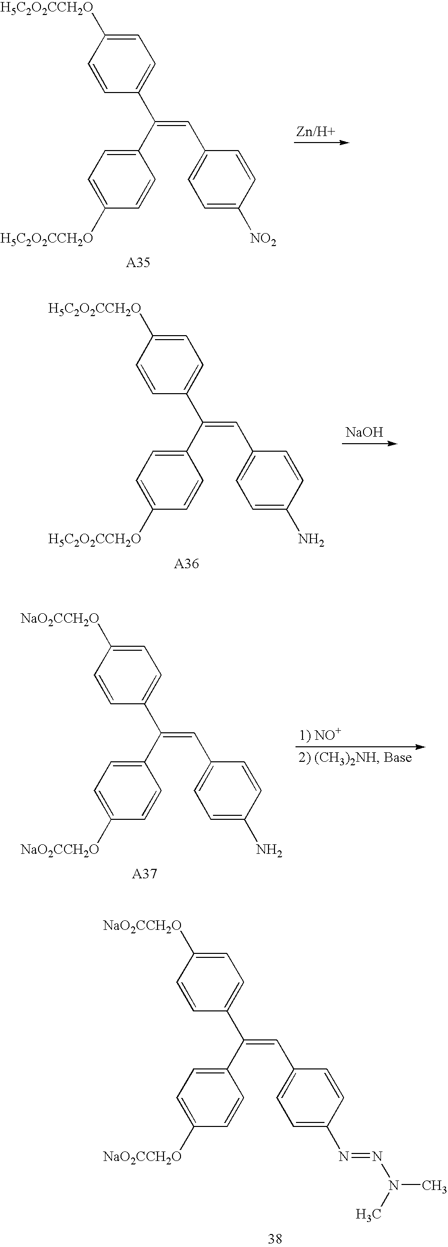

### Example A31

### 1-[Bromo-(4-methylphenyl)-methyl]-4-methylbenzene

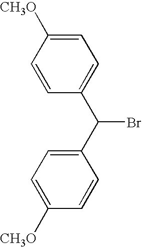

Into a suspension of 100 g of bis-(4-methoxyphenyl)-carbinol and 46 g of calcium chloride in 1.7 l of benzene hydrogen bromide is introduced until saturation. The resulting salt is extracted by suction and the filtrate is reduced.

Yield: 98 g.

### Example A32

### [Bis(4-methoxyphenyl)-methyl]-triphenylphosphoniumbromide

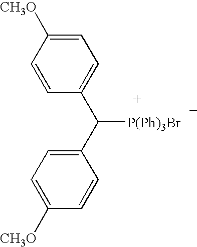

From the bromide from example A31 the title compound is prepared according to example A13.

Yield: 99 g.

### Example A33

### 1-[2,2-Bis(4-methoxyphenyl)-vinyl-4-nitrobenzene

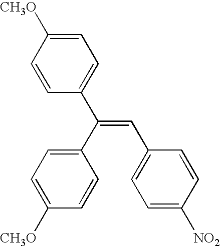

From the phosphonium salt from example A32 and 4-nitrobenzaldehyde the title compound is prepared according to example A17.

Yield: 14 g.

### Example A34

### 4-[1-(4-hydroxyphenyl)-2-(4-nitrophenyl)-vinyl]-phenol

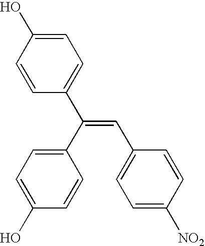

To a mixture of 150 g of the compound from example A33 and 300 ml of pyridine 380 g of conc. hydrochloric acid are added dropwise. Then the reaction solution is heated to 150° C. for 3 hours. Then it is poured onto 2 l of ice water, adjusted to clearly acidic with hydrochloric acid and the title compound is extracted by suction, washed with water and dried.

Yield: 110 g.

### Example A35

### {4-[1-[4-(2-ethoxy-2-oxoethoxy)-phenyl]-2-(4-nitrophenyl)-vinyl]-phenoxy}-ethylacetate

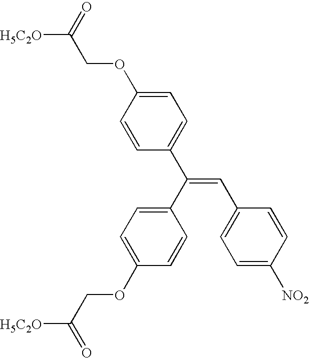

From the phenole from example A34 the title compound is prepared according to example A14.

Yield: 14 g.

### Example A36

### {4-[1-[4-(2-ethoxy-2-oxoethoxy)-phenyl]-2-(4-aminophenyl)-vinyl]-phenoxy}-ethylacetate

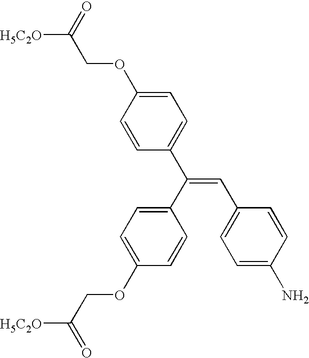

From the nitro-compound from example A35 the title compound is prepared according to example A20.

Yield: 13 g.

### Example A37

### Disodium-(4-{2-(4-aminophenyl)-1-[4-(2-oxido-2-oxoethoxy)-phenyl]-vinyl}-phenoxy)-acetate

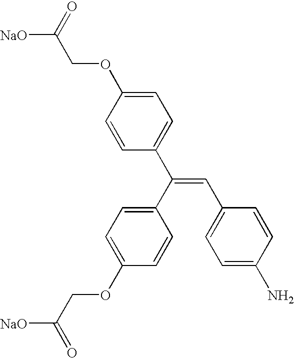

A mixture of 13 g of the ester from example A35, 20 ml of water and 5.3 g of potassium hydroxide is heated for 5 hours under reflux. Then the generated alcohol is removed, the crystalline residue is levigated with 50 ml of water, extracted by suction and dried.

Yield: 10 g.

### Example 38

### Disodium-(4-{2-{4-[(1E)-3,3-dimethyl-1-triazenyl]-phenyl}-1-4-(2-oxido-2-oxo-ethoxy)-phenyl]-vinyl}-phenoxy)-acetate

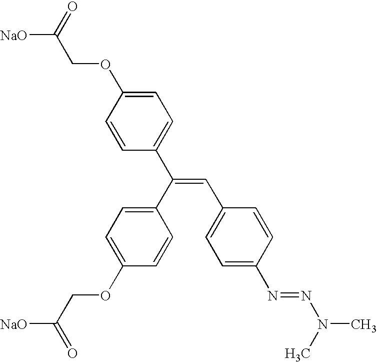

To a mixture of 10 g of disodium salt from example A37, 2 g of sodium hydroxide, 1.7 g of sodium nitrite and 50 ml of water at 0 to 5° C. a solution of 100 ml of water, 130 ml of dimethylformamide and 16 ml of conc. hydrochloric acid is added dropwise. The resulting aqueous diazonium salt solution at 0 to 5° C. is added into a solution of 23 g of sodium carbonate, 14 ml of 40% aqueous dimethylamine solution and 50 ml of water. Then the mixture is reduced to dryness. The residue is dissolved in the necessary amount of water and mixed with 100 ml of saturated aqueous sodium chloride solution. The crystalline precipitating title compound is extracted by suction and dried.

Yield: 5.7 g.

1. Estrogens and anti-estrogens, in which each molecule is core-substituted with at least one dialkyltriazenyl group, with the exception of 4-(3,3-dimethyl-1-triazenyl)-3-methoxy-estra-1,3,5(10)-trien-17-one.

2. Estrogens and anti-estrogens according to claim 1, characterized in, that the substances are derivatives from the group comprising steroids, stilbenes, hexestroles, phenyl-1,2-bis(2,6-dichloro-phenyl)-1,2-bis(ethylenaminoethanes), triphenylethylenes, phenylbenzofuranes, phenylbenzothiophenes, 4,5-bis-phenyl-imidazoles, 2,3-diarylpiperazines and 4,5-phenyl-2-imidazolines.

3. Estrogens and anti-estrogens according to claim 2 of the formula 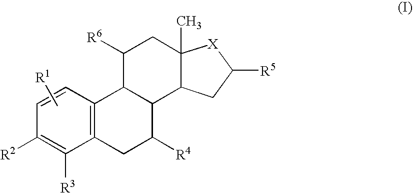
wherein
R¹ is hydrogen, N═N—NR^{7A}R^{7B}, O(CR⁸R⁹)ₙCO₂H, CO₂H or SO₃H,
R² is OH, OCH₃, N═N—NR^{7A}R^{7B} or O(CR⁸R⁹)ₙCO₂H,
R³ is hydrogen, N═N—NR^{7A}R^{7B}, O(CR⁸R⁹)ₙCO₂H, CO₂H or SO₃H,
R⁴ and R⁶ are independently from each other hydrogen, O(CR⁸R⁹)ₙCO₂H, (CR⁸R⁹)ₙCO₂H or C₆H₄OCH₂CO₂H and
R⁴ is moreover (CH₂)₁₀CON(C₁-C₄-alkyl)₂,
R⁵ is hydrogen or OH,
R^{7A} and R^{7B} are independently from each other alkyl,
R⁸ and R⁹ are independently from each other hydrogen, methyl or ethyl,
X is CO, CHOH or C(OH)—C≡CH and
n is an integer from 1 to 10
with the condition, that only one of the residues R¹ to R³ represents N═N≡N—NR^{7A}R^{7B}, and their salts, solvates and solvates of these salts.

4. Estrogens and anti-estrogens according to claim 2 of the formula 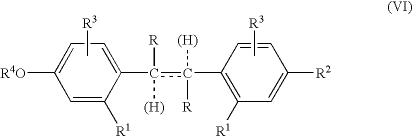
wherein
R is hydrogen, methyl or ethyl,
R¹ is hydrogen, chlorine, methyl, ethyl, CH₂CO₂H, CH(CH₃)CO₂H, OCH₂CO₂H, OCH(CH₃)CO₂H or SO₃H,
R² is OH, OCH₃, OCH₂CO₂H, OCH(CH₃)CO₂H or N═N—NR^{7A}R^{7B},
R³ is hydrogen, chlorine, preferred in position 6, or N═N—NR^{7A}R^{7B},
R⁴ is hydrogen, methyl, ethyl, CH₂CO₂H or CH(CH₃)CO₂H and
R^{7A} and R^{7B} are independently from each other alkyl
with the condition, that either R² or R³ represents N═N—NR^{7A}R^{7B}, and the dashed bonds indicate, that the compounds comprise both ethane as well as ethylene derivatives, and their salts, solvates and solvates of these salts.

5. Estrogens and anti-estrogens according to claim 2 of the formula 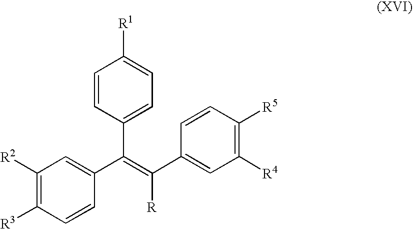
wherein
R is hydrogen, chlorine, chlormethyl or ethyl,
R¹ is OCH₂CO₂H or OCH(CH₃)CO₂H,
R² and R⁴ are independently from each other hydrogen, SO₃H or N═N—NR^{7A}R^{7B},
R³ and R⁵ are independently from each other hydrogen, OH, OCH₃, OCH₂CO₂H, OCH(CH₃)CO₂H or N═N—NR^{7A}R^{7B} and
R^{7A} and R^{7B} are independently from each other alkyl
with the condition, that only one of the residues R² to R⁵ represents N═N—NR^{7A}R^{7B}, and their salts, solvates and solvates of these salts.

6. Estrogens and anti-estrogens according to claim 2, characterized in, that the substances are derivatives of the formula

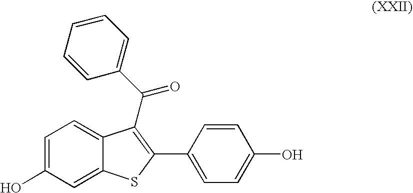

7. Method for the preparation of the compounds according to any one of claims 1 to 6, wherein at least one dialkyltriazenyl substituent is introduced in one or more aromatic rings of an estrogen or anti-estrogen active compound.

8. (canceled)

9. Method for the treatment of tumours of the sexual organs of humans and animals by application of at least one of the compounds according to any one of claims 1 to 6.

10. Process for the preparation of pharmaceuticals against tumours of the sexual organs of humans and animals, comprising combining at least one compound according to any one of claims 1 to 6 with one or more pharmacologically acceptable adjuvants or substrates.

11. Pharmaceuticals, which contain at least one compound according to any one of claims 1 to 6, if necessary together with one or more pharmacologically acceptable adjuvants or substrates.

12. Method for the treatment of tumours of the sexual organs of humans and animals by application of at least one of the pharmaceuticals according to claim 11.

13. Method for the treatment of disease of human and animals by application of at least one of the compounds according to any one of claims 1 to 6.

14. Process for preparation of pharmaceutical against disease of humans and animals, comprising combining at least one compound according to any one of claims 1 to 6 with one or more pharmacologically acceptable adjuvants or substrates.

15. Method for the treatment of disease of humans and animals by application of at least one of the pharmaceuticals according to claim 11.
